(19) United States
(12) Patent Application Publication
(10) Pub. No.: US 2019/0086404 A1
(43) Pub. Date: Mar. 21, 2019

(54) ANALYSIS MEMBRANES FOR MICROFLUIDIC DEVICES, SAID MEMBRANES BEING MADE OF A FIBERGLASS MATERIAL

(71) Applicant: BIOMERIEUX, Marcy l'Etoile (FR)

(72) Inventors: Laurent BOULET, Pollionnay (FR); Frédéric FOUCAULT, Marcy l'Etoile (FR); Christine ROZAND, Saint-Genis-les-Ollieres (FR); Agnès RUBENS, Montceaux (FR)

(73) Assignee: BIOMERIEUX, Marcy l'Etoile (FR)

(21) Appl. No.: 16/083,773

(22) PCT Filed: Mar. 30, 2017
(86) PCT No.: PCT/EP2017/057512

§ 371 Date: Sep. 10, 2018

(30) Foreign Application Priority Data
Mar. 31, 2016 (EP) EP16163345.8

(51) Int. Cl. G01N 33/558; B01L 3/00; B32B 37/20; G01N 30/92

(52) U.S. Cl. CPC G01N 33/558; B01L 3/5023; G01N 30/92; B32B 37/203; B01L 3/5027

(57) ABSTRACT

An analysis membrane of a microfluidic device, said membrane being made in one piece from a liquid-diffusing absorbent material of glass fiber-based composition, said analysis membrane including: at least one zone termed depositing zone, at least one zone termed reaction zone, in which at least one reagent is adsorbed directly to said glass fiber liquid-diffusing material, or indirectly by virtue of a coupling agent, and channels which ensure a fluidic communication between these depositing zone(s) and reaction zone(s), and that at least one reaction zone is circumscribed in a space of the analysis membrane, termed slowed diffusion space, inside which the channels which arrive upstream and/or the channels which depart again downstream: extend into one or more channels of smaller length, and/or extend into one or more channels of smaller width, and/or extend into one or more channels comprising at least one portion having a winding path.

The present invention relates to the general field of microfluidics. It relates more particularly to microfluidic devices for diagnostic purposes, and also to the methods for producing said devices.

Microfluidics can be defined as the study of the phenomena which govern the flow of small volumes of fluid, in particular of liquid. It encompasses the development of systems and devices which make it possible to circulate and/or to manipulate small volumes of fluids, this being for very diverse purposes such as, for example:
for mixing, or even for reacting, all or some of the constituents of one or more fluids, with one another and/or with exogenous compounds,
for separating certain constituents of a fluid, optionally for the purpose of analyzing the chemical and/or physical properties thereof,
for detecting, or even for assaying, target agents which are suspected to be present.
   Microfluidics thus has applications for numerous technical fields.

In the clinical field, diagnostic tests using microfluidic devices have experienced a rapid progression over the past two or three decades (Yetisen et al., 2013-Lab Chip, 2013 (13) 2210-2251: “Paper-based microfluidic point-of-care diagnostic devices”).

Also denoted by “microfluidic sensors”, these devices not only make possible the analysis of small volumes of liquid samples, they also make it possible to undertake, on one and the same analysis platform, a plurality of tests for detecting, or even quantifying, analytes and/or target pathogenic agents, this being by simple and rapid handling.

The present invention focuses more specifically on single-use microfluidic devices, of the type of those which comprise an analysis membrane made of a porous material and which operate according to a principle termed lateral movement of fluids, in the case in point of liquids.

The general principle of the operation of this type of microfluidic device is based essentially on an analysis membrane produced/formed from a sheet of hydrophilic and absorbent porous material, on which and into the body of which is mixed a hydrophilic network formed of zones of interest and of channels. A liquid to be analyzed (for example a liquid biological sample), once deposited on said analysis membrane, can progress, by simple capillary action, inside and through this network of zones of interest and channels, and can be subjected to a qualitative and/or quantitative analysis or a series of qualitative and/or quantitative analyses of its constituents (immunodetection, molecular detection, affinity assay, ligand-receptor binding assay, pH evaluation, etc).

Very schematically, the following may be distinguished on the analysis membranes of the microfluidic devices:
zones termed depositing zones, onto which the liquids to be analyzed are deposited,
zones termed reaction zones, on which the detection and/or assaying reactions are carried out,
optionally, mixed zones which simultaneously act as depositing zones and reaction zones,
optionally, zones termed reservoir zones, in which reagents and/or additives required for the reactions to be carried out are stored, generally in dry form before being rehydrated (or dissolved) and transferred to specific reaction zones,
optionally, secondary depositing zones, which will be fed with the reagent(s) and/or with additive(s), extemporaneously and/or during the analysis process, and
structures forming channels which ensure fluidic conduction between the zones of interest, and through which the liquids progress by a simple phenomenon of capillary action.

Currently, the analysis membranes for microfluidic devices produced from a sheet of hydrophilic and absorbent porous material are formed mainly according to three design modes.

In the first mode, in particular exemplified in WO 2010/102294, WO 2010/003188 and WO 2008/049083, these analysis membranes have a two-dimensional shape which can be described as a full shape and their general geometry is relatively simple (for example, a rectangle, a square, a disk, etc). On these analysis membranes, the hydrophilic networks (depositing zones, reaction zones, mixed zones and/or reservoir zones, fluidic conduction channels) are marked out and delimited by means of structures made from solid and hydrophobic material(s) implanted through the very thickness of the analysis membrane and thus forming liquid-impermeable barriers.

In a second mode, in particular exemplified by US 2008/0317633, the analysis membranes have a two-dimensional shape which can be described as a trimmed or cut form. Such analysis membranes are cut from a sheet of hydrophilic and absorbent porous material, precisely according to the external outline of the hydrophilic network which is formed by the various zones of interest and the fluidic conduction channels.

In a mixed mode, in particular described by Fan et al., 2013 (Nano/Micro Engineered and Molecular Systems (NEMS), 2013-8^{th} IEEE International Conference: “Low-cost rapid prototyping of flexible plastic paper based microfluidic devices”), the analysis membranes have a shape which can be described as a full shape. These analysis membranes are formed/machined from a sheet of hydrophilic and absorbent porous material, the lower face of which is coated with a mechanical reinforcing film. On these analysis membranes, the hydrophilic network is marked out and delimited not by impermeable barriers implanted through the actual thickness of the analysis membrane, but by the void left after removal of porous and hydrophilic material. This removal of material can be carried out by means of very diverse machining techniques, in particular etching, ablation. Nowadays, a laser allows rapid and accurate implementation of these machining techniques.

Regardless of the design mode chosen, because said analysis membranes have for a long time been produced from a cellulose or nitrocellulose fiber sheet, the name “μPAD” (for “microfluidic paper-based analytical devices”) has been widely adopted to refer to microfluidic devices for diagnostic use which integrate such analysis membranes into their structure.

Up until now, this type of material has not made it possible to produce analysis membranes for microfluidic devices which are entirely satisfactory. Because of the extremely small volumes to be handled (barely a few tens of microliters), the liquid samples to be analyzed are very sensitive to drying out. The hygrometry, the temperature, the ambient ventilation are all parameters which influence the rate of drying of the samples. What is more, as the liquid sample spreads through the analysis membrane, the drying rate increases, as does the viscosity, further curbing the diffusion of the sample. According to the conditions under which the analyses are carried out, the samples to be analyzed do not always arrive in sufficient amount at the reaction zones.

In order to overcome this problem, several approaches have been investigated. One of these approaches consists in optimizing the typography of the analysis membranes, in particular to reduce the distances over which the liquids to be analyzed are conveyed to the reaction zones. Another approach aims to improve the performance levels of the existing cellulose and nitrocellulose substrates, for example by functionalizing the fibers, and/or in proposing new fibrous materials with improved properties in terms of hydrophilicity and/or of absorption and/or of fluid conduction.

In this regard, by way of examples of materials that are currently available and known for the production of analysis membranes of microfluidic devices, mention will in particular be made of materials based on fibers of cellulose (including of cotton and of cotton linter), of glass, of silk, of viscose, of polypropylene, of polyester, of polyamide (Nylon®), of poly(lactic acid) or PLA. Said fibers can optionally be functionalized and/or charged and/or doped with additives (for example, talc, diatomite, etc).

In this context, Fang et al., 2014 (Lab Chip, 2014 (14) 911-915: “Paper-based microfluidics with high resolution cut on a glass fiber membrane for bioassays”) describes an analysis membrane, of trimmed shape, entirely made of a liquid-diffusing absorbent material, of glass fiber-based composition. Said membrane is formed according to the desired configuration, by mechanized cutting of a glass fiber sheet by means of a Cricut Expression® cutting machine (Provo Craft @ Novelty, United States), said glass fiber sheet having been previously laminated to a PVC reinforcing film. A glass fiber material is in this case preferred to the (nitro) cellulose fiber materials, for its greater hydrophilicity and its greater wettability, which give rise to a much better liquid conductance and conductivity.

The analysis membrane is configured with a central circular zone, forming a depositing zone intended to receive a liquid sample to be analyzed. Eight channels radiate out from this depositing zone, each channel opening onto a peripheral circular zone of smaller circumference and forming a reaction zone. For the purposes of urine analysis, these reaction zones are prepared for pH evaluation, the detection of glucose, of various proteins, of nitrites, of ketone bodies, etc. The reagents required for the various detections are deposited, in the form of solutions, in the reaction zones which correspond thereto. The membrane takes one hour to dry, under ambient conditions.

The capacity of the glass fiber materials to be able to transfer the liquids with very high speed is described by Fang et al., 2014 as a definite advantage for microfluidics. Nevertheless, when it is a question of designing analysis membranes based on such materials, this high transfer/diffusion speed of the liquids (about 2 mm.s⁻¹) causes difficulties which are difficult to overcome for a person skilled in the art. Indeed, the functionalization of the reaction zones is in this case carried out essentially using adsorption techniques, using reagents in solution in an adsorption buffer. Once the reagents have been deposited on the glass fiber membrane, they are supposed to penetrate the material and to bind to the constituents of the material (essentially fibers and binders), by simple adsorption. The adsorption of the reagents is not instantaneous, but occurs gradually at the rate of the evaporation of the absorption buffer. Given the rapid diffusion of the reagents through the glass fiber membrane and because the reaction zones are open on the rest of the hydrophilic network, it proves to be difficult to delimit the adsorption of the reagents actually inside the reaction zones. The reagents have a strong tendency to diffuse out of the reaction zones before adsorbing into the material. The consequences of these reagent leaks are:
a dilution of the reagents in the reaction zones, and therefore a loss of sensitivity of the detection signal, but also
a risk of contamination of a reaction zone with a reagent originating from a neighboring reaction zone, and a loss of reliability of the analysis device.

Others, such as Songjaroen et al., 2012 (Lab Chip, 2012 (12) 3392-3398: “Blood separation on microfluidic paper based-analytical devices”) or else EP 2 226 635 have opted for composite analysis membranes, that is to say membranes formed from a plurality of parts of various materials, assembled together to form a succession of zones of particular functionality or functionalities. For each of these zones of particular functionality or functionalities, the corresponding material has been specifically selected for particular degrees of hydrophilicity, of porosity and/or wettability. When the materials chosen for the reaction zones exhibit a liquid diffusion speed which is judged to be too high, each reaction zone is independently formed in the material chosen (for example by wax impregnation, by etching or by cutting), then functionalized in isolation from the other constitutive parts of the analysis membrane to be produced. It is only after the binding/adsorption of the reagents that the reaction zones are assembled to the rest of the analysis membrane.

Songjaroen et al., 2012 thus describes a microfluidic device intended for the analysis of plasma proteins using whole blood samples. The analysis membrane is formed by assembling two parts made of different materials; one is cut from a glass fiber sheet (namely, an MF1 filter paper) and the other is cut from a cellulose fiber sheet (in the case in point a Whatman™ No. 1 filter paper). A depositing zone is made in the glass fiber part. At the level of the junction between the two fibrous parts, this depositing zone opens onto a channel made in the cellulose fiber part. This main channel progresses through the cellulose fiber part before dividing into two secondary channels, each opening onto a reaction zone. The zones of interest and the channels of the analysis membrane are marked out and delimited with solid wax, by a wax impregnation method. For the depositing zone, the glass fiber material was chosen for its capacity to be able to filter whole blood and to enable separation between the plasma and the blood cells. The cells are retained in the glass fiber depositing zone, whereas the plasma diffuses out of the glass fiber in the direction of the cellulose fiber part, then through the channels marked out, before reaching the reaction zones. The cellulose fiber material was chosen for its fluidic conduction properties allowing good transfer of the plasma from the depositing zone to the reaction zones.

The microfluidic device described by EP 2 226 635 was designed, for its part, to specifically for the immunodetection of Mycobacterium tuberculosis. The analysis membrane of this device is in the form of a linear succession of parts produced from various hydrophilic and porous materials, cut out and then assembled together so as to form a small strip. According to one particular embodiment, the following are arranged, successively and partially overlapping, from upstream to downstream on the analysis membrane and adhering to the adhesive face of an adhesive tape:
an element forming a depositing zone, capable of receiving a liquid sample to be analyzed; the constituent porous material of this element can be a woven or a nonwoven, made of polyethylene, polypropylene, cellulose, preferentially a filter paper;
a strip of nitrocellulose membrane:
   on the upstream part is an element forming a transient storage zone for a labeled antibody, directed against the MPB64 protein (an analyte indicating the presence of Mycobacterium tuberculosis); this element is made of a nonwoven material of glass fiber-based composition;
   a little further downstream is a capture zone at the level of which an antibody directed against this same MPB64 protein is immobilized in the actual thickness of the nitrocellulose membrane;
an absorbent element made of a material capable of rapidly absorbing a liquid.

Composite analysis membranes, given the independent and isolated production of each of the constituent elements, the optional functionalization of these elements and then the assembly thereof to the rest of the hydrophilic network, prove to be particularly complex and expensive to industrialize.

An objective of the present invention is to overcome the drawbacks encountered in the design and production of analysis membranes for microfluidic devices known at the current time. More particularly, the present invention aims to propose analysis membranes, of novel structure and design, compatible with the constraints of an industrial exploitation of a single-use diagnostic device, in particular in terms of production cost and cost effectiveness.

In this regard, the present invention provides an analysis membrane for a microfluidic device, formed in one piece from a sheet of liquid-diffusing absorbent material, of glass fiber-based composition. Conventionally, said analysis membrane comprises:
at least one zone termed depositing zone,
at least one zone termed reaction zone, in which at least one reagent is adsorbed directly to said glass fiber liquid-diffusing material or indirectly by virtue of a coupling agent,
channels which ensure a fluidic communication between depositing zone(s) and reaction zone(s).

According to the invention, this analysis membrane comprises at least one reaction zone delimited in a space of the analysis membrane, inside which the channels which arrive upstream and/or the channels which depart again downstream:
extend into one or more channels of smaller width, and/or
extend into one or more channels of smaller thickness, and/or
extend into one or more channels comprising at least one portion with a winding path.

Advantageously and according to the invention, at least one of the two faces of the analysis membrane is coated with a mechanical reinforcing layer made of an impermeable hydrophobic material, for example a plastic film of poly(ethylene terephthalate), or PET, type.

Before going further in the description of the invention, the definitions below are given in order to facilitate the understanding and the disclosure of the invention.

The expression “analysis membrane” refers to the main element of a microfluidic device for diagnostic purposes which, by virtue of its various constituent parts, ensures the reception of the liquid sample to be analyzed and the conveying of at least one part of the constituents of this sample to analysis/detection zones, and acts as a structure-support for carrying out these analyses/detections. In the context of the present invention, said analysis membrane is defined as being made in one piece, that is to say that it is made of a single uninterrupted continuous part, made of a single material. This being so, it can be envisioned to add, to an analysis membrane made in one piece according to the invention, added on-elements of secondary functionality or functionalities.

The expression “liquid-diffusing absorbent material” refers to a porous textile of which the structure consists of an ordered or random assembly of fibers (that is to say a woven or a nonwoven). This textile has the capacity not only of absorbing aqueous liquids, but also of diffusing them through its structure. In the context of the present invention, the liquid-diffusing absorbent material used in the production of the analysis membranes is of glass fiber-based composition. For the purposes of simplifying the language, the expression “glass fiber material” can advantageously be used in the present description to denote this type of material. Such glass fiber materials are usually encountered in analysis laboratories (both medical and industrial) where they are commonly denoted “glass fiber filtering media” or “glass fiber filters”, and are conventionally applied to the treatment of liquid compositions, for the purposes of separation and purification. Their production consists in forming sheets/membranes with glass microfibers arranged in a more or less random or ordered manner, and then chemically and/or thermally and/or mechanically interlocking them. By way of examples of such materials, mention may in particular be made of:
Whatman™ filters (GE Healthcare Life Sciences, United States), in particular the MF1, LF1, VF1, VF2 and Fusion 5 papers, intended for the filtration of whole blood and for cell/plasma separation, and the GF/C filter, intended for the separation of solid substances in suspension in a fluid;
filters from the company Sartorius A.G. (Germany), in particular those intended for fluid treatment and analysis.

The term “reagent” is used in the present description in the broad sense to denote any substance used with a view to carrying out one of the analysis or analyses/detection or detections envisioned, whether or not this substance interacts directly with the target(s) sought or intervenes only in an auxiliary capacity. Thus, in the absence of specification, the reagents applied to the present invention can for example be:
in the case of an immunodetection assay: (capture and/or detection) antibodies and/or antigens, haptens, aptamers, optionally coupled to labels, coupling agents, spacer arms, linkers, etc.,
in the case of a molecular detection test: labeled or non-labeled nucleic acids and/or nucleic primers, enzymes (such as enzymes with polymerase, recombinase or helicase activity), labeled or non-labeled nucleic acids, optionally spacer arms, linkers, etc.,
in the case of a direct ionic, chemical or biochemical detection test: chemical reagents, enzymes and enzymatic substrates,
in the case of a microbiological detection test: proteins, antibodies, bacteriophages, antibiotics, enzymatic substrates, etc.

The expression “space where the diffusion speed of the liquids is slowed” refers to a particular portion/part/region of the analysis membrane at the level of which the constitutive glass fiber material of said membrane has been locally modified in its composition and/or in its structure and/or in its configuration in order to slow the diffusion speed of liquids and/or to extend their path between two points, in the case in point between two reaction zones. The time required for a liquid to cover the distance between two points located in such a space is notably greater than the time that said liquid would require to cover this same distance in another portion of the analysis membrane. For the purposes of simplifying the language, the expression “slowed diffusion space” can advantageously be used in the present description to denote such a space made in the analysis membranes according to the invention.

The term “winding path” is intended to mean a non-rectilinear path linking two points and composed of a series of curves and/or segments oriented in various directions. By way of examples of winding paths, mention may in particular be made of waves of elementary shape, such as a sinusoidal, square, triangular or saw-tooth shape (cf. FIG. 17). Paths which are geometrically less regular, without repeated elementary pattern, are also effective. The winding path of the channels at the level of the diffusion spaces makes it possible to artificially increase the distance between two points of the analysis membrane, or rather to increase the temporal distance separating two points of the analysis membrane.

Like Fang et al., 2014, the present invention has opted for analysis membranes made in one piece and the structure of which is entirely made of a glass fiber material.

By creating, in an analysis membrane entirely made of a glass fiber material, slowed diffusion spaces for the liquids and by implanting reaction zones accurately in these spaces, the inventors have managed to overcome a number of problems and difficulties encountered with the membranes proposed by Fang et al., 2014.

Firstly, the problem of diffusion of a reagent out of the reaction zone with which it is associated, and that of the contamination of other neighboring reaction zones by this reagent have been solved. With slowed diffusion spaces, the invention has enabled the containment of each reagent in the reaction zone with which it is associated, and reduced the risk of contamination of the neighboring reaction zones. In order to functionalize the reaction zones, each reagent in solution in an ad' hoc adsorption buffer is deposited in its reaction zone where it is held for a sufficiently long period of time to be able to adsorb thereto. Forced drying (non-passive drying, for example by heat treatment and/or by ventilation) makes it possible to even further limit the dispersion of the reagents outside the reaction zones.

Certain embodiments of the slowed diffusion spaces according to the invention give rise to an effective reduction in the diffusion speed of the liquids. The diffusion speed of water at ambient temperature (about at 20-28° C.) can in fact be divided by a factor at least equal to 2, compared with the diffusion speed on the same support of glass fiber-based composition, before modification. Such embodiments offer better control of the concentration/amount of reagents finally adsorbed in the reaction zones, and make it possible to increase the contact time between the analytes of the sample to be tested and the reagents present in each reaction zone located in these slowed diffusion spaces. The resulting improvement in the quality of the reactions is all the more significant for the analyses which require sequential treatment of the liquid sample; the reaction zones dedicated to such an analysis are arranged in series, each of the reaction zones being specifically functionalized so as to be able to carry out a particular step of the analysis process.

Finally, by virtue of its design, an analysis membrane according to the invention imposes, on the liquids to be analyzed, large variations in speed in their diffusion. These large variations in diffusion speed form, just upstream of the slowed diffusion spaces, a bottleneck at the level of which the liquid to be analyzed accumulates. This temporary accumulation makes it possible to reduce the surface area of liquid exposed to gas exchanges and, consequently, to significantly slow down the drying of the liquid to be tested.

In a first embodiment of the slowed diffusion spaces of an analysis membrane according to the invention, the glass fiber material, in this particular part of the analysis membrane, has inlays of solid wax.

Given the great structural brittleness which characterizes glass fiber materials and given their relatively large thickness (generally between 200 and 1000 μm), the inventors have applied a particular technique of wax impregnation in order to create such inlays of solid wax in a thickness of a glass fiber sheet.

This particular technique is described in detail in European patent application No. EP16163217.9, filed by the applicant on Mar. 31, 2016. According to one preferred procedure, the latter is carried out schematically in the following way:
the following are printed, in solid ink of wax-comprising composition, on a transfer film:
   a first intermediate pattern intended to coat the lower face of a glass fiber sheet which will constitute the basic structure of the analysis membrane; this first intermediate pattern is like the slowed diffusion space to be produced;
   a second intermediate pattern intended to coat the upper face of the glass fiber sheet; said pattern is an inverted image of the first intermediate pattern;
   the first and second intermediate patterns can be printed on one and the same transfer film or on two independent transfer films;
these intermediate patterns (with their transfer film) are affixed directly in contact with the two faces of the glass fiber sheet such that, on either side of the thickness of this glass fiber sheet, they are mutually symmetrical, at least substantially facing one another;
maintained at least substantially horizontally, said glass fiber sheet and said transfer film(s) are subjected to a heat treatment capable of causing at least partial melting of the constituent wax of the intermediate patterns, and to a mechanical treatment capable of momentarily compressing the thickness of all or part of said glass fiber sheet;
said glass fiber sheet is subjected to a mechanical and thermal expansion phase, capable of allowing said sheet to at least partly return to its initial thickness and allowing the wax to resolidify within the thickness of said glass fiber sheet.

In order for the wax inlays thus produced to be able to slow the flow of the liquids to be analyzed without blocking the passage thereof, the printing of the intermediate patterns is carried out with a solid ink with a low concentration of wax and/or in a pale color (color of weak intensity). The intensity of the color printed is proportional to the amount of wax deposited on the transfer film; this color intensity is chosen so that, once in the thickness of the glass fiber sheet, the amount of wax thus transferred is insufficient to completely block the passage of the liquids by capillary action, but just sufficient to significantly lower the speed of this passage.

By way of indication, this printing of wax is carried out using a printer of Xerox® ColorQube™ type, fed with solid inks having the references Xerox® 108R00931 (cyan color), 108R00932 (magenta color), 108R00933 (yellow color) and 108R00934/108R00935 (black color).

In a second embodiment of the slowed diffusion spaces of an analysis membrane according to the invention, precisely in the particular regions of the analysis membrane which correspond to the slowed diffusion spaces, channels or microchannels of small width and/or of small thickness are formed in the glass fiber material; these microchannels channel the flow of the liquids which pass through said space. The average cross section of these microchannels is advantageously between 10 000 μm² and 500 000 μm², preferentially between 1000 μm² and 150 000 μm², and even more preferentially between 30 000 μm² and 55 000 μm².

When a Whatman™ MF1 filtering medium is used to produce an analysis membrane according to the invention, the channels of the slowed diffusion spaces advantageously have a width of between 150 μm and 1.5 cm, preferentially between 200 and 1000 μm.

According to this first embodiment and this second embodiment, a reduction in the diffusion speed of the liquids is obtained by virtue of a reduced hydrophilicity and/or a reduced density of flow. In so doing, in a slowed diffusion space according to the invention, the diffusion speed of water (subjected to ambient temperature, that is to say a temperature of about 20-28° C.) is divided by a factor at least equal to 2, compared with its diffusion speed on the same support of glass fiber-based composition, before modification.

In a third embodiment of a slowed diffusion space according to the invention, the channels progressing inside these spaces comprise at least one portion which has a winding path. The winding path of these channels makes it possible to extend the fluidic distance which separates two points of the same analysis membrane.

Advantageously and according to the invention, said winding path can be graphically represented as a wave of elementary shape chosen from: a sinusoidal, square, triangular or saw-tooth wave or a combination thereof.

Regardless of the embodiment of the slowed diffusion spaces, whether it is by inlay of solid wax, by the establishing of (micro)channels of small width and/or of small thickness, and/or the establishing of (micro)channels comprising portions with a winding path, an analysis membrane according to the invention advantageously comprises, located in a slowed diffusion space, at least two reaction zones. These reaction zones are arranged in direct fluidic communication, one behind the other.

According to one variant of implementation of the invention, the reaction zones of one and the same slowed diffusion space are arranged in indirect fluidic communication, one behind the other and inserted between which is at least one buffer zone. Said at least one buffer zone is not functionalized. The glass fiber material, at this level, has undergone no treatment, nor any modification in its structure or in its composition. Like the reaction zones, the buffer zones of a slowed diffusion space of an analysis membrane according to the invention have a surface area generally of between 0.5 mm² and 25 mm², preferentially of about 1-10 mm². When, on the basis of the general shape of said buffer zones, a width and a length or a base and a height can be defined, then the corresponding widths and lengths or the corresponding bases and heights, are of the same order of magnitude, generally in a ratio of 1 to 5.

Advantageously, in the slowed diffusion spaces of an analysis membrane according to the invention, the distance between two adjacent zones, reaction zones and/or buffer zones, is less than 15 mm, preferentially less than 5 mm, and even more preferentially is about 1-2 mm.

In doing so, the various reaction zones can be functionalized concomitantly with a minimal risk of contamination from one reaction zone to another, and good control of the amount of reagents loaded into each reaction zone.

Advantageously, a slowed diffusion space of an analysis membrane according to the invention comprises at least two reaction zones placed in direct fluid communication, one behind the other and at a distance of less than 5 mm, preferentially at a distance of about 1-2 mm.

An analysis membrane according to the invention may be of full shape. To this effect, on the base of a glass fiber sheet (optionally cut beforehand to the shape and to the final dimensions of the analysis membrane), the outline of the various depositing and reaction zones and of the channels ensuring the fluidic communication is advantageously drawn with solid wax. The solid wax implanted through the thickness of the glass fiber sheet forms liquid-impermeable barriers. Likewise, to this effect, the slowed diffusion spaces advantageously integrate inlays of solid wax. European patent application EP16163217.9 (bioMérieux) teaches a method for producing such a full-shape analysis membrane.

An analysis membrane according to the invention can also be of trimmed shape. To this effect, said analysis membrane is cut from a sheet of glass fiber material precoated with a mechanical reinforcement layer on one of its faces, according to the external outline of the hydrophilic network formed by the various depositing and reaction zones, the fluidic conduction channels and the slowed diffusion spaces.

Finally, an analysis membrane according to the invention can also be of mixed shape. To this effect, it is formed from a sheet of glass fiber material, one of the faces of which, termed lower face, is precoated with a mechanical reinforcement layer and the outline of the hydrophilic network formed by the depositing and reaction zones, the fluidic conduction channels and the microchannels of the slowed diffusion spaces is etched in the body of the glass fiber material until it reaches the surface of the mechanical reinforcement layer.

The present invention also relates to an analysis membrane for a microfluidic device and also to a microfluidic device comprising such an analysis membrane, characterized, in combination, by all or some of the features above or below.

Other objectives, features and advantages of the invention will emerge on reading the following examples which refer to the appended figures and in which:

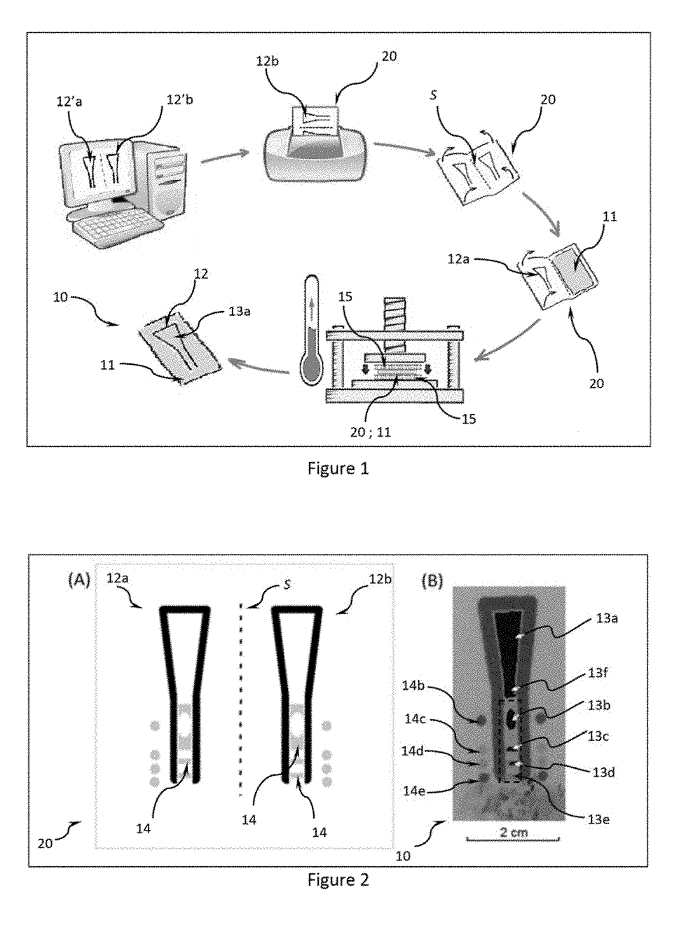

FIG. 1 is a diagrammatic representation of a wax impregnation method which makes it possible to form an analysis membrane in accordance with a first embodiment; in this first embodiment, the analysis membrane is said to be of full shape and the slowed diffusion spaces are created by wax inlay;

FIG. 2 sets out, in (A), a diagrammatic representation of a transfer film used in the wax impregnation method illustrated in the preceding figure, and in (B), a photograph of an analysis membrane of full shape obtained from this transfer film;

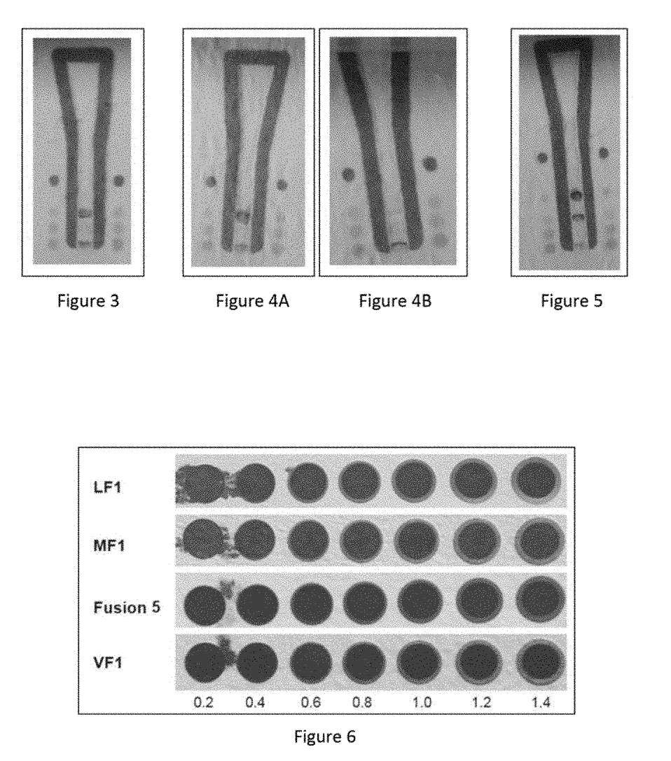

FIG. 3, FIG. 4A and FIG. 4B are photographs illustrating the application of an analysis membrane of full shape according to the invention, for immunodetection purposes;

FIG. 5 is a photograph of an analysis membrane according to the invention formed for the screening for dengue virus, by codetection of the NS1 and DomIII proteins;

FIG. 6 shows photographs of filtering media etched with a CO₂ laser and illustrating the influence of the width of the etching lines on the leaktightness of the patterns;

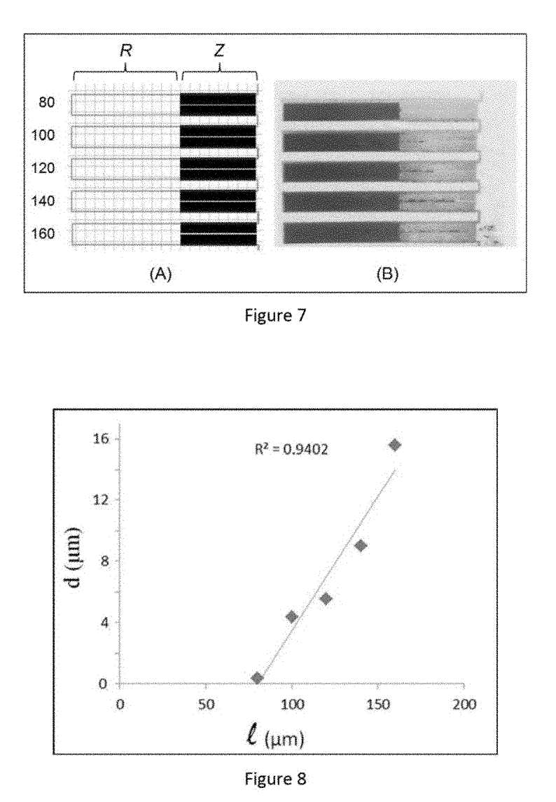

FIG. 7 sets out a graphic representation (A) and a photograph (B) illustrating the influence of the width of the microchannels on the flow distance of liquids through a slowed diffusion space made in a glass fiber filtering medium;

FIG. 8 is a graphic representation showing the correlation between the width of the microchannels and the flow distance of the liquids through a slowed diffusion space, made in a glass fiber filtering medium;

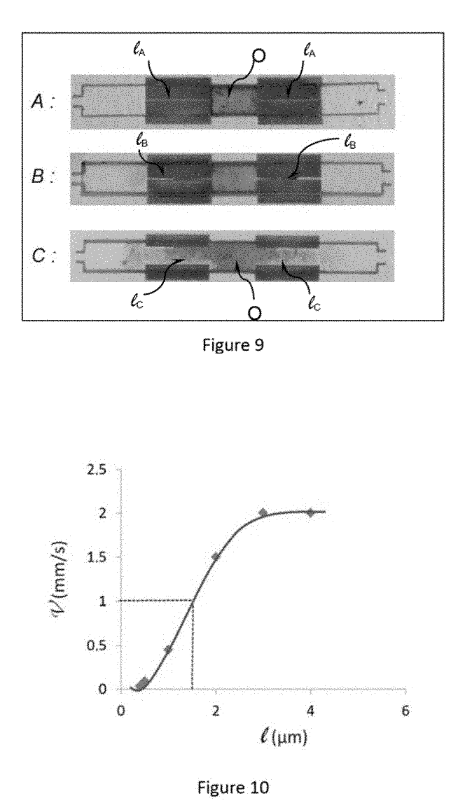

FIG. 9 sets out photographs illustrating the influence of the width of the (micro)channels on the flow speed of the liquids through a glass fiber filtering medium;

FIG. 10 is a graphic representation showing the change in the flow speed of the liquids as a function of the width of the (micro)channels formed in a glass fiber filtering medium;

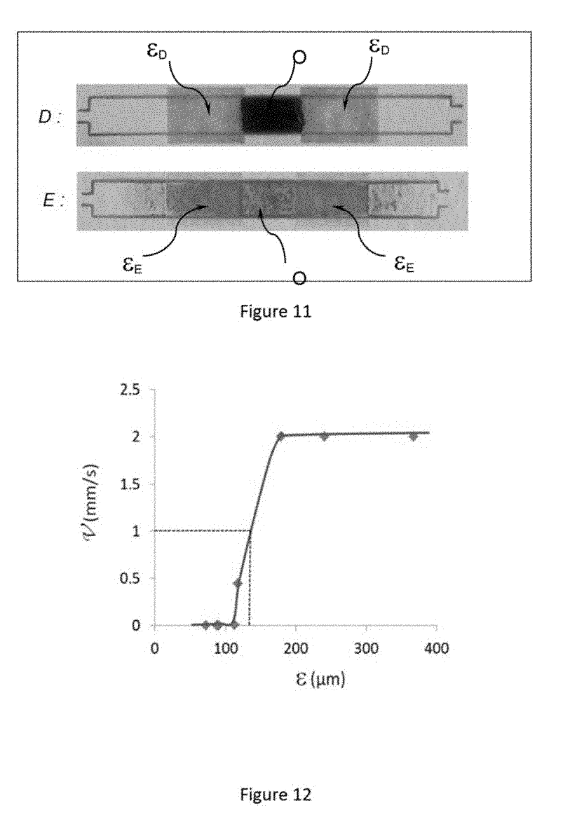

FIG. 11 sets out photographs illustrating the influence of the thickness of the filtering medium on the flow of the liquids;

FIG. 12 is a graphic representation showing the change in the flow speed of the liquids as a function of the thickness of the filtering medium;

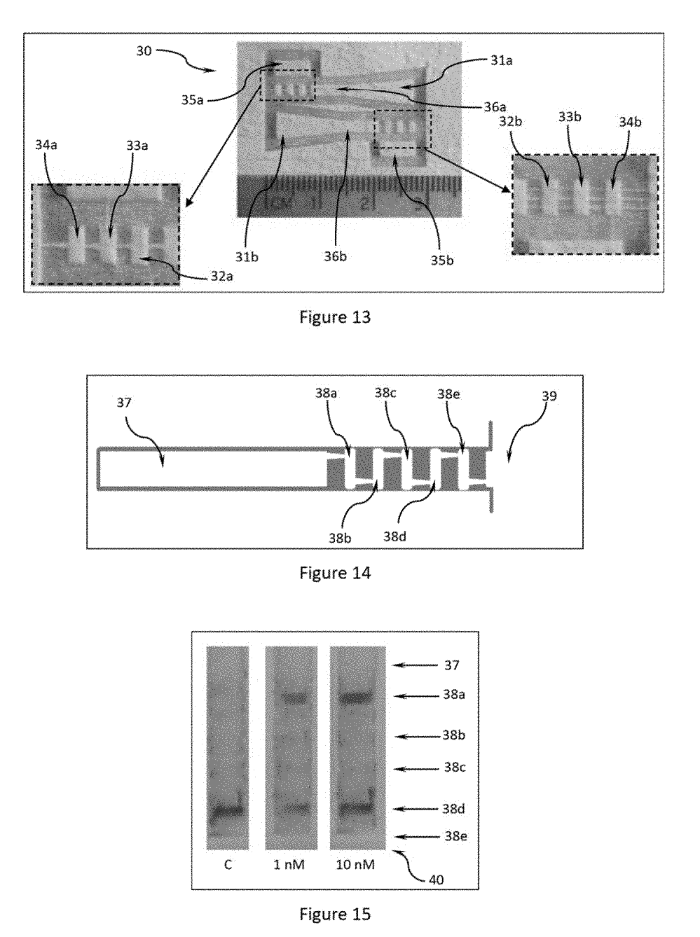

FIG. 13 is a photographic representation of an example of an analysis membrane according to the invention, formed by laser/ablation etching, with magnification of the slowed diffusion spaces;

FIG. 14 is a graphic representation illustrating an example of an analysis membrane according to the invention, formed by ablation of hydrophilic material and functionalized with a view to a molecular detection of target nucleic sequences;

FIG. 15 shows three photographs setting out the results of a molecular detection of a target nucleic sequence, by means of an analysis membrane according to the invention;

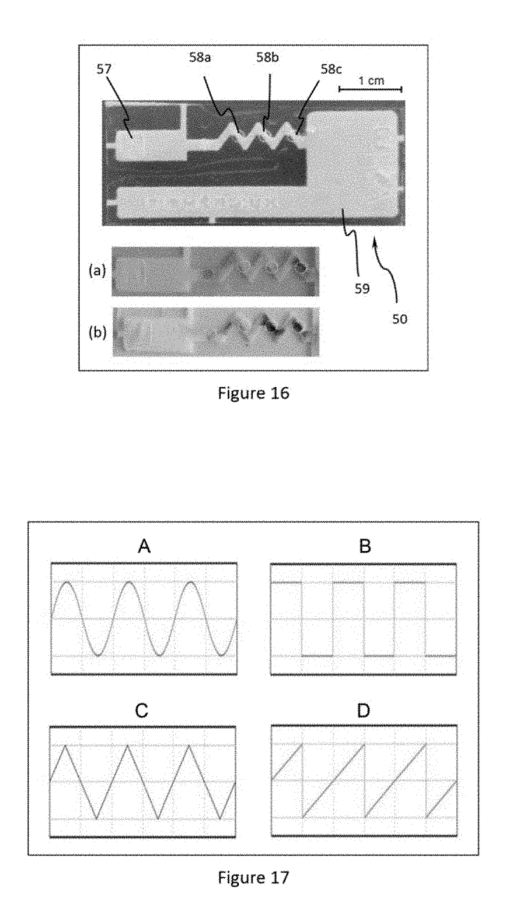

FIG. 16 is a photographic representation of another example of an analysis membrane according to the invention, formed by laser ablation, with magnification of the slowed diffusion spaces;

FIG. 17 is a graphic representation of waves of elementary shapes such as sinusoidal, square, triangular and sawtooth shapes, that the winding paths within the meaning of the present invention can optionally have.

### EXAMPLES

I. Production of an Analysis Membrane of Full Shape According to the Invention, by Impregnation with Solid Wax
a) Implementation of the wax impregnation method which is the subject of European patent application No. EP16163217.9

FIG. 1 illustrates diagrammatically the implementation of a method for producing an analysis membrane 10, in accordance with a first embodiment of the invention according to which the analysis membrane is of full shape and the slowed diffusion spaces are obtained by wax inlay.

In a first step, an image 12′a corresponding to the pattern to be integrated into the thickness of a glass fiber sheet 11 is computer generated by means of drawing software. This first image 12′a is duplicated in a symmetrical image 12′b.

By means of a solid ink printer, the two images 12′a and 12′b are printed on a transfer film 20, so as to form two intermediate patterns 12a and 12b arranged symmetrically relative to an axis S. This axis of symmetry S is also printed on the transfer film 20, by way of visual marker. FIG. 2(A) illustrates in detail such a transfer film 20 on which the two intermediate patterns 12′a and 12′b are printed.

Once this printing has been carried out, the transfer film 20 is folded in half along the axis of symmetry S, the intermediate patterns 12a and 12b facing inward. The latter are thus superimposed on one another. A glass fiber sheet 11 is slipped inside the folded transfer film 20, inserted between the two intermediate patterns 12a and 12b.

The glass fiber sheet 11, sandwiched between the two flaps of the transfer film 20, is thus pressed between two horizontal compression/heating plates and two rubber parts 15 forming a thermal and mechanical pack.

The assembly is subjected to a pressure of about 1 kg/cm² at a temperature of 120° C., for approximately 3 minutes. During this process, the wax previously printed on the transfer film 20 is transferred onto the two faces of the glass fiber sheet 11, then impregnates the thickness thereof.

The printing is carried out with a printer of Xerox® ColorQube™ type, fed with solid inks of references Xerox® 108R00931 (cyan color), 108R00932 (magenta color), 108R00933 (yellow color) and 108R00934/108R00935 (black color). The transfer film 20 is a sheet of ordinary office paper.

As illustrated in FIG. 2(A), the intermediate patterns 12a and 12b printed on the transfer film 20, of rectilinear general shape, have an upper part which widens out and an open-ended narrower lower part. Their external outlines are printed with black ink with a high color intensity. In so doing, the corresponding amount of wax proved to be sufficient to allow the forming and the obtaining of impermeable edges in the thickness of the glass fiber sheet.

The upper part of the intermediate patterns 12a and 12b opens out and is intended to form a hydrophilic zone, in the case in point a depositing zone 13a capable of receiving a liquid sample to be analyzed. Once deposited in the depositing zone 13a, the liquid sample will be able to migrate by capillary action in the direction of the other hydrophilic zones of the device, namely to the lower part where the reaction zones are located. FIG. 2(B) is a photograph of an analysis membrane 10 prepared from a transfer film 20 (after a particular functionalization of the reaction zones and after use).

This lower part of the pattern corresponds to a slowed diffusion space according to the invention. This space, marked on FIG. 2(B) by a box with a discontinuous outline, is obtained by transfer and impregnation of a layer of solid ink 14 of pale color (weak intensity) applied to the two faces of the glass fiber sheet 11. The intensity of the color to be applied is determined so as to reduce the hydrophilicity of the glass fiber material without blocking the passage of the liquids.

Located within this slowed diffusion space are four reaction zones 13b, 13c, 13d and 13e, spared by the impregnation with wax. The position of each of these zones of interest is specifically marked by the marking elements 14b, 14c, 14d and 14e, also marked out with the colored solid ink.

Once the waxes have been transferred into the thickness of the glass fiber sheet 11, the various more or less hydrophilic zones will be able to be functionalized and/or loaded with reagents as a function of the desired analyses and tests.

b) Examples of Functionalization of the Analysis Membrane 10

### 1. Functionalization for Hepatitis B Screening

According to a first mode of application of the analysis membrane 10 previously described, said membrane was functionalized for hepatitis B screening, by immunodetection of the HBs antigen contained in the blood.

In this context, particular functions were allocated to the zones of interest 13b, 13c, 13d and 13e.

The zone 13d is functionalized by means of anti-HBs monoclonal antibodies specific for the reaction; the zone 13d forms the “spot test”. The zone 13e is functionalized by means of anti-alkaline phosphatase monoclonal antibodies specific for the detection conjugate; the zone 13e forms the “positive control spot”. The zone 13c is functionalized by means of antibodies non-specific for the reaction (for example, anti-rat antibodies); the zone 13c forms the “negative control spot”.

The functionalization of these various zones with the antibodies is carried out by antibody adsorption.

Above these three spots, the zone 13b is dedicated to the storage of the second part of the conjugated complex (biotin-labeled anti-HBs monoclonal antibodies); the zone 13b forms the “anti-HBs-biot Ac spot”.

Optionally, the depositing zone 13a can also be used for storage of the conjugate of the immunoenzymatic reaction (streptavidin-alkaline phosphatase or STRE-ALP) in dried form. This conjugate will be redissolved by the liquid phase of the sample to be analyzed.

### 2. Functionalization for Dengue Virus Screening

According to a second mode of application of the analysis membrane 10 previously described, said membrane was functionalized for the detection, in blood and plasma, of two dengue virus proteins: the NS1 protein and the domain III of the envelope protein of the virus (DomIII).

For the detection of the NS1 protein, the following deposits were carried out:
zone 13e (anti-ALP control zone): 0.35 μl of an anti-alkaline phosphatase antibody 1 mg/ml in PBS,
zone 13c (test zone): 0.35 μl of an anti-NS1 antibody at 1 mg/ml in PBS,
zone 13b: 1 μl of a solution of alkaline phosphatase-labeled anti-NS1 antibodies at the concentration of 50 μg/m1 in PBS-0.5% BSA,
zone 13d (negative control zone): 0.35 μl of PBS-0.5% BSA.
   Once the deposits have been carried out, the analysis membrane is allowed to dry for 3 minutes at 60° C. It is then ready to be used.

To test this analysis membrane, 20-25 μl of plasma to which 1 μg of NS1 protein has been added are deposited in the reservoir zone 13a. Once all of the sample has penetrated and migrated within the analysis membrane, 10 μl of BCIP/NTP (bromo-4-chloro-3-indole phosphate/4-nitroblue tetrazolium chloride) are added to the zone 13b. The anti-ALP control zone and the test zone very rapidly become positive, the negative control zone remaining colorless (cf. FIG. 3). The detection is carried out in 5-6 minutes. In parallel, the experiment is carried out with 20-25 μl of NS1-free plasma. In this case, only the control zone becomes positive.

Similarly, the analysis membrane 10 previously described was functionalized for the detection of the dengue virus envelope protein, using capture and detection antibodies directed against DomIII. The corresponding analysis membrane was tested. A positive detection of DomIII and a negative detection are illustrated by FIG. 4A and FIG. 4B, respectively.

Finally, the codetection of the NS1 and DomIII proteins were also successfully carried out by means of an analysis membrane (multiplexing), the structure and the design of which are very similar to those of the previous analysis membranes 10. The difference is based on the presence of an additional reaction zone in the slowed diffusion space. A photograph of this new analysis membrane according to the invention is presented in FIG. 5.

II. Production of an Analysis Membrane of Mixed Shape According to the Invention, by a CO₂ Laser Ablation Technique
a) Determination of the thickness of the edges of the patterns to be etched in a glass fiber material in order to obtain good leaktightness.

CO₂ laser ablation tests (Speedy 100 platform, from the company Trotec®, Austria) were carried out on various fiberglass materials of the filtering medium type with a view to determining the thickness of the edges to be applied to the patterns for the analysis membranes according to the invention.

To this effect, for each of the filtering media tested, the lower face was covered beforehand with a liquid-impermeable, mechanical reinforcement layer, in the case in point an adhesive film made of poly(ethylene terephthalate). On the upper face, a series of seven circular patterns was laser-etched with an etching depth sufficient to reach the mechanical reinforcement layer. The etched circles have one and the same internal diameter of 10 mm, whereas their etched edges are of variable thicknesses, ranging from 0.2 mm to 1.4 mm, in increments of 0.2 mm. A drop of dye is deposited on the center of the patterns in order to verify their leaktightness.

Table 1 below presents the characteristics of the filtering media tested and also the operating parameters applied to the CO₂ laser used.

TABLE 1

| | LF1 | MF1 | Fusion 5 | VF2 |
|---|---|---|---|---|
| | (GE Health- | (GE Health- | (GE Health- | (GE Health- |
| | care Life | care Life | care Life | care Life |
| | Sciences) | Sciences) | Sciences) | Sciences) |
| Thickness of | 247 | 367 | 370 | 785 |
| the medium | | | | |
| (μm) | | | | |
| Laser power | 30 | 30 | 30 | 30 |
| (W) | | | | |
| Laser move- | 3.55 | 3.55 | 1.42 | 1.42 |
| ment speed | | | | |
| (m · s⁻¹) | | | | |

The results obtained are presented in FIG. 6, in the form of photographs of the etched filtering media.

Whatever the filtering medium used, it is possible to generate leaktight patterns by virtue of the laser etching method. It is advisable to optimize for each glass fiber medium, as a function in particular of the fiber density and of the thickness of the material, the power and movement speed of the laser, and also the width of the lines etched.
b) Determination of the influence of the width of the microchannels on the flow distance of the liquids

CO₂ laser ablation tests were carried out on a glass fiber filtering medium of Whatman™ Mf1 type (thickness 367 μm) with a view to determining the influence of the width of the microchannels of a slowed diffusion space according to the invention on the flow distance of the liquids.

With reference to FIG. 7, a series of patterns comprising a reservoir R linked to a slowed diffusion zone Z, comprising a microchannel of variable width from one pattern to the other, were etched by CO₂ laser. The widths tested are 80, 100, 120, 140 and 160 μm. A constant volume of dye (20 μl) is then deposited in the reservoir zone. The distance traveled by the dye in the channel is measured in each of the cases. The results are presented in numerical form in Table 2 below and in graphic form in FIG. 8.

TABLE 2

| Microchannel width (μm) | Distance traveled (mm) |
|---|---|
| 80 | 0.4 |
| 100 | 4.4 |
| 120 | 5.5 |
| 140 | 9.0 |
| 160 | 15.6 |

The distance (d) traveled by the dye in the microchannels is proportional to the width (l) thereof. It is thus possible to control the routing of the liquids by virtue of the laser etching method described herein. Moreover, the implementation of such an analysis makes it possible, for a given glass fiber material, to evaluate the width of the microchannels and the distance between two reaction/buffer zones to be applied to a slowed diffusion space of an analysis membrane according to the invention.
c) Determination of the influence of the width of the (micro)channels on the flow speed of the liquids

The nominal speed of migration of a liquid with a viscosity close to water in the glass fiber support of Whatman™ MF1 type (thickness 367 μm) is about 2 mm/s.

A study was carried out on a glass fiber filtering medium of Whatman™ MF1 type (thickness 367 μm) with a view to determining the influence of the width of the (micro)channels on the flow speed of the liquids.

In this regard, glass fiber membranes, the lower face of which is reinforced with a PET film, were formed with a CO₂ laser, by ablation of material so as to reveal (micro)channels of various widths. The diffusion rate of a low-viscosity liquid such as a dye diluted in water (η=1 mPa·s, at ambient temperature), through these various (micro)channels was then measured.

FIG. 9 shows three of the membranes thus prepared, denoted A, B and C. These membranes have a central zone O in which the colored liquid was deposited, and the diffusion of this liquid through the (micro)channels l_{A}, l_{B} or l_{C}, arranged on either side of the central zone O, was monitored. The width of the microchannels l_{A} of the membrane A is 100 μm, that of the microchannels l_{B} of the membrane B is 400 μm, and that of the channels l_{C} of the membrane C is 3 mm. FIG. 10, in the form of a curve, presents the change in diffusion rate V of the colored liquid (η=1 mPa·s) as a function of the width e of the (micro)channels. The measurements were carried out at ambient temperature (about 20° C.).

It is thus considered that a channel with a width of about 1.5 mm makes it possible to reduce by half the circulation speed of the liquid. A channel with a width of less than 1.5 mm makes it possible to significantly reduce the diffusion rate of the liquids through the glass fiber support of Whatman™ MF1 type.
d) Determination of the influence of the thickness of the (micro)channels on the flow of the liquids

The study of the glass fiber membranes was continued by analyzing the influence of the thickness of the material on the flow speed of the liquids.

In this regard, glass fiber membranes, the lower face of which is reinforced with a PET film, were formed with a CO₂ laser, by ablation of material so as to create zones of smaller thickness. The application of a laser makes it possible to eliminate a given percentage of the thickness of the material, this thickness varying with the power and the speed of the laser. If the power/speed ratio is too high, the etching is too deep and tends toward total elimination of the material. If the power/speed ratio is too low, the impact of the etching on the containment of the liquids is negligible. By means of an experiment, evaluating the speed of a liquid through a laser-etched zone, as a function of the residual thickness of the membrane (ε), it is possible to determine the optimal etching conditions which result in acceptable containment of the liquids.

FIG. 11 shows two of the membranes thus prepared, denoted D and E. These membranes have a central zone O in which the colored liquid (η=1 mPa·s, at ambient temperature) was deposited, and the diffusion of this liquid through two laser-etched zones over a length of 10 mm, on either side of the central zone O, was monitored. The etched zones ε_{D} of the membrane D have a residual thickness of about 120 μm. The residual thickness of the etched zones ε_{E} of the membrane E is about 170 μm. These residual paper thicknesses on the etched zones were measured using a comparator, a mechanical instrument which makes it possible to determine very small thicknesses.

FIG. 12, in the form of a curve, presents the change in diffusion rate V of the colored liquid (η=1 mPa·s) as a function of the residual thickness ε of the etched zones. The measurements were carried out at ambient temperature (about 20° C.). The maximum thickness of paper of MF1 type which allows efficient retention of the liquids, that is to say a decrease by half in the speed of the liquids within it, is around 130 μm.

III. An Example of an Analysis Membrane Formed by Laser Ablation/Etching

As shown in FIG. 13, the analysis membrane 30 exemplified comprises two hydrophilic analysis networks placed head-to-tail, each hydrophilic network comprising:
a sample-depositing zone 31a or 31b,
a slowed diffusion space housing three reaction cross zones 32a or 32b, 33a or 33b, and 34a or 34b, aligned one behind another; the distance between two adjacent reaction zones is about 1 mm,
a secondary depositing or additional storage zone, 35a or 35b, placed in direct fluidic communication with the reaction zone 33a or 34b, through a microchannel.

In the slowed diffusion space of the top hydrophilic network, each reaction zone is in direct fluidic communication with the adjacent reaction zone, through a single microchannel of conical shape.

In the slowed diffusion space of the bottom hydrophilic network, each reaction zone is in direct fluidic communication with the adjacent reaction zone, through three parallel microchannels of rectilinear general shape.

IV. An Example of Molecular Detection of Target Nucleic Sequences on an Analysis Membrane According to the Invention Formed by Laser Ablation/Etching

FIG. 14 shows an etching pattern according to a particular configuration of an analysis membrane 40 according to the invention, formed in a glass fiber material of Whatman™ MF1 type, with a view to detection of target nucleic sequences.

The corresponding analysis membrane 40 comprises, upstream, a mixed zone 37, mixed in the sense that it serves, on the one hand, to pre-store the dry reagents and, on the other hand, to receive the liquid reagents (sample and enzymatic substrate) during the analysis. Downstream is a slowed diffusion space within which is located a series of reaction zones 38a-38d, positioned like the rungs of a ladder and connected to one another by microchannels. These microchannels have a beveled shape, with a maximum width of about 300 μm. The third zone 39, at the exit of the slowed diffusion space, serves as a capillary pump for the flow of the liquids.

In this particular analysis membrane model, the channel which provides the fluidic communication between the zone 37 and the reaction zones of the slowed diffusion space structurally merges with a part of the zone 37 and constitutes an extension thereof up to said slowed diffusion space.

The zone 37 is functionalized by drying of the following detection reagents, in a buffer which allows easy redissolving thereof by the sample: i) biotin-labeled oligonucleotides specific for the single-stranded DNA target to be detected, ii) horseradish peroxidase-labeled oligonucleotides specific for another part of the sequence of the DNA target and iii) alkaline phosphatase-labeled streptavidin.

The reaction zones 38a-38e are used for the specific capture and the visualization of the presence of the DNA target. The first reaction zone 38a is functionalized by adsorption of monoclonal antibodies directed against horseradish peroxidase (specific test). The following three reaction zones 38b, 38c and 38d are functionalized by adsorption of BSA. They are used here as a negative control but could also allow multiplexed detections. The last reaction zone 38e is functionalized by adsorption of BSA-biotin (positive control).

At the end of the reaction, a coloration of the specific test band, the intensity of which depends on the concentration of DNA target in the sample, is observed.

FIG. 15 illustrates the results of a detection using such an analysis membrane. From left to right, the concentrations of target nucleic acid sequences are 0 (negative control), 1 nM and 10 nM.

V. Another Example of an Analysis Membrane Formed by Laser Ablation/Etching

As shown in FIG. 16, the analysis membrane 50 was formed from a glass fiber material of Whatman™ MF1 type, by CO₂ laser ablation cutting. The analysis membrane 50 comprises:
a sample-depositing zone 57,
a slowed diffusion space in which three reaction cross zones 58a, 58b and 58c are aligned one behind the other,
a zone 59, at the outlet of the slowed diffusion space, which serves as a capillary pump for the flow of the liquids.

The slowed diffusion space corresponds here to a portion of conduction channel at which the channel has a width of about 1.0-1.5 mm and a winding path. This path has the shape of a triangular elementary wave. The time taken by the liquids to pass through the various reaction zones 58a, 58b and 58c is thus regulated, on the one hand, by the small width of the channel and, on the other hand, by its winding path.

In order to stiffen the structure and to facilitate handling during production and the validation tests, the analysis membrane 50 is covered with a transparent adhesive film, applied to its lower face. The assembly is surmounted by a rigid polymer plate (about 1 mm thick). For experimental purposes, this plate is transparent and is made of polymethyl methacrylate, or PMMA. The underlying analysis membrane thus remains entirely visible, facilitating the visual monitoring of the diffusion of the liquid to be analyzed through the analysis membrane.

For the final diagnostic device, it may be envisioned that the analysis membrane is contained in a case made of an opaque polymer material. The upper face of this case may optionally have the configuration of the transparent PMMA plate, which comprises cut out openings, positioned facing the zones of interest of the analysis membrane, in particular the sample-depositing zone 57, and the reaction zones 58a, 58b and 58c.

By way of example of particular functionalization of the analysis membrane 50:
the reaction zone 58b corresponds to a test zone,
the reaction zone 58a corresponds to a negative control zone, and
the reaction zone 58c corresponds to a positive control zone.

In this regard and as illustrated in FIG. 16, the part (a) shows the visual of a test with a negative result and the part (b) shows the visual of a test with a positive result.

1. An analysis membrane of a microfluidic device, formed in one piece from a sheet of liquid diffusing absorbent material of glass fiber-based composition, said analysis membrane comprising:
at least one zone termed depositing zone,
at least one zone termed reaction zone, in which at least one reagent is adsorbed directly to said glass fiber liquid-diffusing material, or indirectly by virtue of a coupling agent,
channels which ensure a fluidic communication between depositing zone(s) and reaction zone(s),
wherein at least one reaction zone is circumscribed in a space of the analysis membrane, termed slowed diffusion space, inside which the channels which arrive upstream and/or the channels which depart again downstream:
extend into one or more channels of smaller width, and/or
extend into one or more channels of smaller thickness, and/or
extend into one or more channels comprising at least one portion with a winding path.

2. The analysis membrane as claimed in claim 1, wherein at least one of its two faces is coated with a mechanical reinforcement layer made of impermeable hydrophobic material.

3. The analysis membrane as claimed in claim 1, wherein said microchannels have an average cross section advantageously of between 10 000 μm² and 500 000 μm².

4. The analysis membrane as claimed in claim 1, wherein, in the slowed diffusion space, the glass fiber material has solid inlays made of wax.

5. The analysis membrane as claimed in claim 1, wherein it comprises, in a slowed diffusion space, at least two reaction zones placed in direct fluidic communication, one behind the other and at a distance of less than 15 mm.

6. The analysis membrane as claimed in claim 1, wherein it comprises, in a slowed diffusion space, at least two reaction zones placed in indirect fluidic communication, one after the other and between which is inserted at least one buffer zone.

7. The analysis membrane as claimed in claim 5, wherein the distance between two adjacent zones, reaction zones and/or buffer zones, is less than 5 mm.

8. The analysis membrane as claimed in claim 1, wherein it has a shape termed trimmed shape; said analysis membrane being cut from a sheet of glass fiber material by following the external outline of the network formed by the various depositing and reaction zones, the fluidic conduction channels and the slowed diffusion spaces.

9. The analysis membrane as claimed in claim 1, wherein it is formed from a sheet of glass fiber material, one of the faces of which, termed lower face, is coated with a mechanical reinforcement layer, and in that the outline of the network formed by the depositing and reaction zones, by the fluidic conduction channels and by the microchannels of the spaces where the diffusion rate of the liquids is slowed, is etched in the body of the glass fiber material until it reaches the surface of the mechanical reinforcement layer.
